(19) United States
(12) Patent Application Publication
(10) Pub. No.: US 2003/0180368 A1
(43) Pub. Date: Sep. 25, 2003

(54) Production of microparticles

(75) Inventors: Gordon Findlay Dawson, Glasgow (GB); Frank Koppenhagen, By Kilmarnock (GB)

Correspondence Address: FOLEY HOAG, LLP, PATENT GROUP, WORLD TRADE CENTER WEST, 155 SEAPORT BLVD, BOSTON, MA, 02110, US

(73) Assignee: Cenes Drug Delivery Limited, Kilmarnock (GB)

(21) Appl. No.: 10/317,547

(22) Filed: Dec. 12, 2002

Related U.S. Application Data
(63) Continuation of application No. 09/646,200, filed on Nov. 9, 2000, now abandoned.
Application No. 09/646,200 is a 371 of international application No. PCT/GB99/00685, filed on Mar. 15, 1999.

(30) Foreign Application Priority Data
Mar. 14, 1998 (GB) GB 9805417.4

(51) Int. Cl. A61K 9/16; B01J 13/04; B01J 13/02; A61K 9/50

(52) U.S. Cl. 424/490; 264/4.1

(57) ABSTRACT

A method of forming microparticles (especially including a pharmaceutically active agent) comprising incorporating an emulsifier in an aqueous liquid or in a non-aqueous solution (e.g. of dichloromethane) containing a polymer; forming a dispersion of the aqueous liquid in the non-aqueous solution; and agitating and adding a non-solvent (e.g. silicone oil) for the polymer. The presence of the emulsifier allows microparticle production over a wide range of conditions.

### FIELD OF THE INVENTION

The present invention relates to a method of producing microparticles for use in delivering a pharmaceutically active substance, particularly a peptide, protein or polynucleotide, and to the microparticles themselves and pharmaceutical compositions thereof.

### BACKGROUND

In particular, the present invention relates to the so-called phase separation method of producing microparticles wherein an emulsion of water droplets is formed in a continuous phase comprising a polymer dissolved in a non-aqueous solvent such as dichloromethane. The polymer is caused to coacervate out of solution around the water droplets by the addition of a non-solvent for the polymer, such as a silicone oil. The process is generally carried out under vigorous agitation using a mixer to prevent coalescence of the droplets or incipient microparticles.

However, a problem of this method is that the so-called stability window (i.e. the relative ratios of the components of the mixture e.g. silicone oil non-solvent, polymer, water and non-aqueous solvent which results in the successful formation of microparticles) is rather small. This limits the practical application of the method. For example, when a ternary diagram is drawn representing silicone oil, polymer and dichloromethane solvent there is only a small area representing a very restricted range of these components (typically around 36% silicone oil, 4% polymer and 60% dichloromethane) which results in microparticles. It would be desirable to have a method of producing microparticles which was operable over a wider range of concentrations, in particular so that it could be adapted for use with a variety of active substances of varying molecular weight, solubility, polarity etc.

Also, it would be useful to be able to employ higher amounts of polymer or water, leading to higher loadings of active substance, higher yields of microparticles and increased drug entrapment.

Further, the so-called water-in-oil emulsion used to prepare the microparticles is an unstable emulsion and vigorous agitation is required in order to maintain a small droplet size and prevent coalescence during the polymer precipitation phase separation step. Typically, a high viscosity silicone oil is used as the non-solvent (since low viscosity oils tend to result in an even more reduced stability window). The vigorous agitation or mixing of the system leads to the generation of large amounts of heat, which can be undesirable where the pharmaceutically active material to be loaded into the microparticles is a heat sensitive material, such as a peptide or protein. Also, heat tends to promote evaporation of volatile non-aqueous solvent (e.g. dichloromethane) leading to lack of process control. To overcome this problem, the art has previously suggested the use of cooling systems, such as dry ice or liquid nitrogen; but such cooling methods not preferred for large scale industrial application.

A selection of prior art references relating to the phase separation method of producing microparticles is as follows. Patent specification U.S. Pat. No. 3,531,418 describes the production of a polymer solution at high temperature. As the solution cools, the polymer is precipitated out of solution in the form of microparticles around a solid active agent core. Solid active agents may need to be ground to size, which may generate heat and denature heat-sensitive active agents. Moreover, use of a non-aqueous solvent in direct contact with the active agent can also result in denaturation. Alternatively, aqueous solutions may be encapsulated. U.S. Pat. No. 4,166,800 also relates to the preparation of microspheres by low temperature (−40° to −100° C.) phase separation of a polymer and a core material. U.S. Pat. No. 4,389,331 employs cooling to room temperature as the phase separation step.

Patent specification U.S. Pat. No. 4,622,244 describes a standard phase separation method wherein phase separation is brought about by the addition of a phase separation agent, such as a polymeric material or a non-solvent for the polymer being used to produce the microparticles. Phase separation occurs either at low temperatures of at least −30° C. or at room temperature. However, isolation of the microparticles has to be carried out at a temperature of −30° C. or lower. U.S. Pat. No. 4,673,595 describes hardening of the microparticles at a temperature of between 0° and 25° C. employing particular aliphatic fluorinated or fluorohalogenated hydrocarbons as hardening agents. Silicone oil non-solvent is used as a phase separation agent.

Patent specification W089/03678 describes the use of a second non-solvent such as heptane to harden the microspheres prior to collection thereof. Patent specifications EP0377477 and U.S. Pat. No. 5,066,436 describe the use of other hardening agents such as fatty acid esters. Patent specifications U.S. Pat. No. 5,000,886 and U.S. Pat. No. 5,500,228 describe the use of volatile silicone oils as hardening agents, so as to facilitate removal thereof so that there is no residue in the microcapsules.

Patent specifications GB2234896 and U.S. Pat. No. 5,603,960 disclose the use of surfactants during microparticle production; Patent specification GB2234896 discloses the use of a hardening mixture comprising heptane and Span 80 surfactant oil-in-water emulsion to harden the microspheres and to remove non-encapsulated active peptide material so as to avoid an initial burst of active when the microparticles are administered to a patient. Patent specification U.S. Pat. No. 5,603,960 describes a reversal of the standard phase separation technique, wherein the aqueous dispersion is formed in silicone oil non-solvent, and the solution of polymer in dichloromethane is added thereto to initiate phase separation. There is a suggestion that Span 40 surfactant may be included in the aqueous dispersion of active in the silicone oil non-solvent.

An object of the present invention is to provide a process having a broad stability window.

### SUMMARY OF THE INVENTION

The present invention, is based on the use of a emulsifier to stabilise the system.

Specifically, the present invention provides a method of producing microparticles, which comprises;

incorporating an emulsifier in an aqueous liquid and/or a non-aqueous solution containing a polymer;

forming a dispersion of the aqueous liquid in the non-aqueous solution; and

agitating the dispersion and adding thereto a non-solvent for the polymer so as to form polymer microparticles.

Thus, the present invention includes a emulsifier in the aqueous liquid which forms the aqueous discontinuous phase of the “water-in-oil” dispersion and/or in the continuous “oil” phase; and is present at the interface between the aqueous droplets and the non-aqueous solvent. The use of a emulsifier has been found to expand the stability window, and so to provide a process which is capable of producing microparticles over a relatively wide range of conditions (especially quantities of non-aqueous solvent, aqueous liquid, polymer, non-solvent and active substance). It allows larger quantities of water and of polymer to be included in the dispersion. The process is thus robust and adaptable to a variety of active substances. In particular, the process should be suitable for the sustained release of small peptide molecules. It also enables lower viscosity non-solvents to be employed, which may not be possible in the absence of the emulsifier.

The use of a emulsifier has been found to allow non-solvents and agitation conditions to be used, which are such that undue heat is not generated during the production of the polymer microparticles thereby protecting any pharmaceutically active agent incorporated therein from thermal degradation. Generally, the aqueous liquid comprises a pharmaceutically active agent suspended or dissolved therein. In principal, the pharmaceutically active agent can be any active solid or liquid substance but the method of the present invention is particularly applicable to those active agents which are susceptible to thermal degradation at temperatures above room temperature (i.e. 20° C.). Particularly preferred active agents are proteins and peptides, such as enzymes, hormones, antigens etc. and those which exert a therapeutic or prophylactic effect, or can be used as diagnostic agents. The peptides or proteins may be recombinant, synthetic or from natural sources. Typically the peptide or protein may be lysozyme, insulin, thyrotropin releasing hormone (TRH), luteinising hormone releasing hormone (LHRH) or analogues thereof (e.g. Leuprolide), or cytochrome C. Such proteins and peptides have been found not to be denatured by the formulation process.

The emulsifier is usually incorporated in the aqueous liquid or non-aqueous solvent by being dissolved or emulsified therein. The emulsifier is usually present in an amount of up to 60% by weight, generally up to 30% by weight, preferably up to 20% by weight, typically 5-15% by weight of the liquid. There is generally at least 1% by weight of emulsifier present. Preferably, the emulsifier is a non-ionic surfactant, such as those having a hydroxyl-containing hydrophilic portion and a long chain fatty acid lipophilic portion. Typical non-ionic surfactants are available under the trademarks Span, Tween and Brij. Span type materials are partial esters of the common fatty acids (lauric, palmitic, stearic, and oleic) and hexitol and anhydrides (hexitans and hexides), derived from sorbitol. Tween type materials are derived from the Span materials by adding polyoxyethylene chains to the non-esterified hydroxyl groups. Span products tend to be oil-soluble and dispersable or insoluble in water; while Tween products are soluble or well dispersed in water. Brij surfactants include polyoxyethylene ester groups. Preferred non-ionic surfactants are Span 20, 40, 60, 65 and 80. Anionic surfactants or cationic surfactants (such as quaternary ammonium compounds) may also be used. The HLB value of the surfactant is normally in the range 2 to 9.

The emulsifier (which may or may not be a surfactant) may be any pharmaceutically acceptable emulsifying agent and may be non ionic, for example gum arabic, alginic acid, cetostearyl alcohol, cetyl alcohol, a glucose fatty acid ester, glyceryl monooleate, glyceryl monostearate, hydroxypropyl cellulose, a medium chain triglyceride, low molecular weight methylcellulose, a poloxamer, a polyoxyethylene alkyl ether, a polyoxyethylene castor oil derivative, a polyoxyethylene fatty acid ester, a polyoxyethylene stearate, polyvinyl alcohol, a sorbitan fatty acid ester, or a sucrose fatty acid ester; cationic, for example cetrimide, monoethanolamine or triethanolamine; or anionic, for example a cholic acid derivative, carbomer, docusate sodium oleic acid, propylene glycol alginate, sodium dodecyl sulfate, stearic acid, white wax or yellow wax; or mixtures of the above.

The aqueous liquid, optionally containing pharmaceutically active agent, is dispersed in a non-aqueous solution containing a polymer, generally a pharmaceutically acceptable polymer for pharmaceutical applications. Generally, the dispersion will be unstable and requires vigorous agitation. The degree of agitation may be determinative of the droplet size of the discontinuous aqueous phase. The surfactant also has an effect in controlling particle size. The aqueous liquid is usually 0.3-50%, generally 5-50%, especially 10-20% by weight of the dispersion. A preferred range is 1-20% by weight.

The polymer used for forming the polymer microparticles is generally a pharmaceutically acceptable polymer such as a polyester, polyvinylchloride, polycaprolactone or the well known polylactide family of polymers, in particular a polylactide-co-glycolide polymer. In these polymers, the ratio of lactide to glycolide (and endcapping) and molecular weight may be varied and determines the rate of release of the active material from the microparticles. The molar ratio of lactide to glycolide can vary in the range 100:0 to 0:100. However, molar ratios of 100:0 to 50:50 are preferred since the copolymers tend to be soluble in the non-aqueous solvents preferably employed. The ratio is preferably between 70:30 and 35:65 (more preferably 70:30 to 50:50). Preferred copolymers have a lactide to glycolide ratio of 50:50 or 75:25.. The number average molecular weight Mn of the polylactide polymer may be in the range 5,000 to 50,000. The inherent viscosity (i.v.) is generally from below 0.2 up to 8. In vivo, such polymers undergo biodegradation by random, non-enzymatic scission to form lactic acid and glycolic acid metabolites. Thus, the bulk degradation of the polymer is determinative of the release time for most of the active agents to be included within the microparticle, such that the microparticles have a sustained release effect. The sustained release period may be up to 365 days but will generally be in the range 5 to 100 days, typically 10 to 30 days. The use of an emulsifier according to the present invention has been found to produce microparticles of narrow size distribution and excellent consistency of shape. Clearly, it is desirable that the particles should have a consistent spherical shape, which are easier to inject, rather than irregular shapes.

The non-aqueous solvent for the polymer solution is generally an organic solvent. The polymer may be present in an amount of up to 25% by weight, preferably in the range 0.5 to 10% by weight, more preferably 1 to 3% by weight. Dichloromethane (methylene chloride) is conventionally employed as it is volatile and easily removed from the final microparticles. It has been found to be especially beneficial in the present invention.

In the phase separation stage, a non-solvent for the polymer is added to the continuous non-aqueous phase in order to coacervate the polymer from solution onto the dispersed aqueous droplets. The dispersion is kept in a constant state of agitation in order to prevent coalescence of the aqueous droplets or of the forming microparticles. If the non-aqueous solution has a relatively high viscosity, then the vigorous agitation results in the production of considerable heat, which must be removed by cooling in conventional processes. In a preferred feature of the present invention, the non-solvent is a material of relatively low viscosity which is dissolvable in the non-aqueous solvent but does not substantially increase the viscosity thereof. The temperature of the method can be kept to around room temperature and is preferably in the range 10-25° C. However, no external cooling means are required in order to achieve this, thereby substantially facilitating the industrial application of the process.

Typically, the non-solvent is a silicone oil, such as those available from Dow Corning or Fluka Chemicals (Gillingham, UK). The Dow Corning 200 series of oils are particularly preferred. Preferably, the viscosities lie in the range 50-150 mPa, although viscosities of up to 500 may be employed. In contrast, the prior art has generally employed silicone oils having viscosities of 500 to 1000 mPa and above.

The polymer microparticles formed may then be hardened in conventional manner, e.g. by admixing with a non-solvent for the polymer such as a liquid hydrocarbon e.g. heptane or other conventional non-solvents. Agitation may be continued to prevent coalescence of the microparticles until hardening is completed.

Thereafter, the microparticles may be filtered and washed. Generally, the microparticles are then dried, which has the effect of removing residual non-aqueous solvent and residual water so as to leave the microparticle consisting substantially of polymer, active agent if present and residual emulsifier (e.g. in amounts up to 5% by weight, more generally up to 2% by weight). The residual emulsifier is beneficial in that it facilitates the resuspension of the microparticles in water where the mioroparticles are to be used in the form of an aqueous suspension, for example as an injectable sustained release pharmaceutical formulation.

It has been found that using the process of the present invention loading efficiencies for active substance of at least 40%, typically at least 60% and even at least 80% has been achieved. The loading efficiency is defined as the amount of active substance entrapped in the microparticle divided by the amount introduced into the process.

The process is suitable for the production of both small (down to 0.5 g) and large (e.g. 10 g) batches of microparticles.

In a further aspect of the present invention, there is provided microparticles comprising a pharmaceutically active agent, a emulsifier and a pharmaceutically acceptable polymer. Because of the method of production, the microparticles are substantially free of non-solvent, such as silicone oil. The substantial freedom of the microparticles from hydrophobic non-solvents such as silicone oil, together with the presence of emulsifier ensures that the microparticles of the present invention are particularly easy to resuspend in water in order to provide pharmaceutical compositions thereof. Typical active agents are as follows.

| | Candidate Active Agents |
|---|---|
| Class | Sub Class |
| Antibacterials | Beta Lactams |
| | Tetracyclines |
| | Aminoglycosides |
| | Macrolides |
| | Metronidazole |
| | 4-Quinolones |
| Anticoagulants | Epoprostenol |
| Antidepressants | Tricyclics |
| | Serotonin Specific Reuptake |
| | Inhibitors |
| Antifungals | Miconazole |
| Antipsychotics | Chlorpromazine |
| | Haloperidol |
| | Benperidol |
| Cytotoxics | |
| Hormones | Thyroxine |
| | Insulin |
| | Somatropin |
| | LHRH |
| | TRH |
| | Calcitonin |
| | Bromocriptine |
| Hormone analogues | Cyproterone acetate |
| | Leuprolide |
| | Goserelin |
| | Naferelin |
| Immunosupressants | Azathioprine |
| | Cyclosporin |
| | Tacrolimus |
| | Interferons |
| Non Steroidal Anti | Diclofenac |
| Inflammatories | |
| Peptides & Proteins | Interleukins |
| | Interferons |
| | Colony stimulating Factors |
| | Growth Factors |
| | Chemokines |
| | Bradykinins |
| | Neurotransmitters |
| Sex Hormones | Oestrogen |
| | Progesterone |
| | Stilboestrol |
| | Medroxyprogesterone acetate |
| Steroids | Hydrocortisone and its esters |
| | Beclomethasone and its esters |
| | Fludrocortisone and its esters |

LHRH = Luteinising Hormone Releasing Hormone
TRH = Thyrotrophin Releasing Hormone

Thus, a further aspect of the present invention provides pharmaceutical formulations, particularly for injection, comprising the microparticles suspended in an aqueous pharmaceutically acceptable liquid.

In fact, the microparticles of the present invention may also find use in non-pharmaceutical areas such as agriculture (e.g. for the sustained release of pesticides) or in food (e.g. to incorporate sustained release flavourings into chewing gum) and this forms a further aspect of the invention.

As well as generalised systemic release of active agent, the microspheres can be used for targetting active agents to specific tissue sites. Drug targetting applications arise from the ability of microspheres to immobilise a depot of a pharmacologically active material in a tissue bed. This can be achieved either by direct injection into the tissue or by administration of particles having the correct size distribution into the arterial system supplying the tissue of interest, the particles then being trapped in the relevant capillary bed.

Generally, the median particle size Dv50 (also known as Dv0.5) of the microparticles is in the range 2-300 microns, preferably 10-300 microns, more preferably 10-100 microns and particularly 10-50 microns.

The Dv50 is the particle diameter having 50% of the total sample volume above and below it. It therefore represents the median volume diameter. This is relevant to active agents delivery systems, since particle volume is related to the quantity of active agent that can be loaded.

It is found that the loading efficiences of the active agent in the microparticles of the present invention is good and is generally in the range 5-75%.

### DETAILED DESCRIPTION OF PREFERRED EMBODIMENTS

The invention will now be described by way of example only with reference to the following examples.

### EXAMPLE 1

(No Active)

Three grams of a 10% (w/v) aqueous Span 20 surfactant emulsion were transferred into a glass vial. To this vessel was added 25 ml of 3% (w/v) poly(D,L-lactide-co-glycolide) (50/50 molar, i.v. −0.7) in dichloromethane. The mixture was homogenised for 1 minute using a Silverson homogeniser. Whilst continuing mixing, 15, 20, 25, 30, 35 or 40 ml of silicone oil (DC 200 of viscosity about 110 mPa.s) obtained from Fluka chemicals, Dorset UK was added at a rate of 2 ml per minute using a syringe driver equipped with a 50 ml glass syringe. Hereafter, mixing was continued for 1 minute. The resulting mixture was then added to 200 ml stirred n-heptane, and stirring continued for a minimum of 30 minutes. Stirring was halted, and the formed microparticles were allowed to settle. The supernatant was decanted, and another 200 ml of n-heptane added. Stirring was continued for at least a further 30 minutes. Hereafter, stirring was stopped, and the microparticles collected on a cellulose ester filter (1.2 μm pore size). The resulting microparticles were allowed to airdry overnight. Mean microparticle size was determined by laserlight diffraction, and is shown in Table 1.

TABLE 1

| Volume of silicone oil added (ml) | Particle size (μm) |
|---|---|
| 15 | 51 |
| 20 | 45 |
| 25 | 54 |
| 30 | 232 |
| 35 | 261 |
| 40 | 254 |

Methodology for Determining Particle Size:

Aliquots of dried microparticles were resuspended in de-ionised degassed water. After brief sonication of the sample, the particle size was determined by laser-light diffraction using a Malvern Mastersizer S equipped with a stirred cell. Particle sizes were expressed as the median of the volume distribution DVO-5.

### EXAMPLE 2

(Alternative Emulsifiers)

I) This example illustrate the use of different emulisifers (i.e.surfactants). The procedure of Example 1 was repeated, with the only change that different emulsifiers were used (Span 20,40 and 65), and the volume of silicone oil was 20 ml. The resulting mean particle sizes are shown in Table 2A.

TABLE 2A

| Emulsifier used | Particle size (μm) |
|---|---|
| Span 20 | 67 |
| Span 40 | 104 |
| Span 65 | 219 |

II) 600 mg emulsifier was dispensed into a mixing vessel along with 25 ml 3% w/v poly(D,L-lactide-co-glycolide) (50:50 ratio, i.v. −0.7) in dichloromethane, and 3 ml of demineralised water. The system was then homogenised and 20 ml of silicone oil (100 mPas) added under mixing. The resulting mixture was transferred into 200 ml n-heptane and stirred for 1 hour after which the product was allowed to settle. The supernatant was decanted, 200 ml fresh n-heptane added, and stirring continued. The resulting product was then collected on a membrane filter.

Following drying, the product was assessed for the presence of microparticulates. Microparticles were sized by the method of laser light diffraction and the results summarised in Table 2B. This data illustrates that a range of emulsifiers can be utilised in the process.

TABLE 2B

| | Product particle |
|---|---|
| Emulsifier | Diameter (μm) |
| Span 80 | 48 |
| 1-Monomyristoyl-rac-glycerol | 118 |
| 1-Monooleoyl-rac-glycerol | 31 |
| oleylamine | 126 |
| Decanoyl-N-methyl glucamide | 158 |
| 6-O-Palmitoyl-L-ascorbic acid | 58 |

### EXAMPLE 3

(Various Oil Viscosities)

Example 3 to shows the effect of different oil viscosities and mixing speeds. The procedure described in Example 1 was repeated, with the only change that 20 ml of silicone oil of viscosity 110 or 378 m.Pa.s was added, whilst the mixing speed was varied (6500, 8600 and 11,500 rpm). The resulting mean particle sizes are shown in Table 3.

These data illustrate the importance of silicone oil grade with respect to the microparticle particle size. Low viscosity oils have the potential to produce smaller particles than their more viscous counterparts. Furthermore, mixing speed was demonstrated to affect particle size such that increased speeds resulted in smaller particles. These results reflect the importance of the intermediate emulsion with respect to the characteristics of the final product.

TABLE 3
Particle sizes in μm

| | Mixer speed (rpm) | | |
|---|---|---|---|
| oil, viscosity (m · Pa · s) | 6,500 | 8,600 | 11,500 |
| 110 | 210 | 95 | 72 |
| 378 | 375 | 264 | 235 |

### EXAMPLE 4

(Entrapment of Bovine Serum Albumin)

Two ml of a 20% (w/v) aqueous emulsion of Span 20 and 1 ml of an aqueous solution of bovine serum albumin (BSA) were transferred into a glass vessel. To this vessel, 25 ml. of 3% (w/v) poly(D,L-lactide-co-glycolide) (50/50 molar RG505, i.v.−0.7 or 75/25 molar RG755, i.v.˜0.6) in dichloromethane was added. The mixture was homogenised for 1 minute using a Silverson homogeniser. Under continuous mixing, 20 ml of silicone oil (silicone oil DC200, −100 mPa.s) was added at a rate of 2 ml per minute using a syringe driver equipped with 50 ml glass syringe. Hereafter, mixing was continued for 1 minute. The resulting mixture was then added to 200 ml stirred n-heptane. Stirring was continued for a minimum of 30 minutes, whereafter stirring was stopped, and the formed microparticles allowed to settle. Supernatant was decanted, another 200 ml of n-heptane added, and stirring continued for at least 30 minutes. Hereafter, stirring was stopped, and the miocroparticles were collected over a cellulose ester filter (1.2 μm pore size). The microparticles were dried under vacuum overnight. Microparticle sizes were determined using laserlight diffraction, and the entrapment of BSA in the microspheres determined using a bicinchoninic acid (BCA) protein assay. The results are shown in Table 4.

TABLE 4

| | BSA loading | Particle size | | BSA loading |
|---|---|---|---|---|
| Polymer | level (% w/w) | (μm) | Recovery | efficiency (%) |
| RG505 | 9.1 | 131 | 83 | 17.5 |
| RG505 | 6.3 | 130 | 86 | 24.3 |
| RG505 | 2.9 | 87 | 84 | 33.4 |
| RG505 | 1.0 | 120 | 91 | 26.0 |
| RG755 | 9.1 | 82 | ND | 19.5 |
| RG755 | 6.3 | 69 | ND | 8.4 |
| RG755 | 2.9 | 44 | ND | 6.6 |
| R6755 | 1.0 | 61 | ND | 4.0 |

ND = not done

### EXAMPLE 5

(Entrapment of Lysozyme)

Two ml of a 20% (w/v) aqueous emulsion of Span 20 and 1 ml of an aqueous solution of 75 mg/ml lysozyme were transferred into a glass vessel. To this vessel, 25 ml. of 1% (w/v) poly(D,L-lactide-co-glycolide) (50/50 molar, i.v.−0.7) in dichloromethane were added. The mixture was homogenised for 1 minute using a Silverson homogeniser. Under continuous mixing, 10 ml of silicone oil (silicone oil DC200,˜100 mPa.s) was added at a rate of 2 ml per minute using a syringe driver with a 50-ml glass syringe. Hereafter, mixing was continued for 1 minute. The resulting mixture was then added to 200 ml stirred n-heptane. Stirring was continued for a minimum of 30 minutes, after which the microparticles were washed and collected as described in Example 4. Mean microparticle size was determined by laserlight diffraction and found to be 17 μm. The entrapment of lysozyme in the microspheres was determined using a BCA protein assay. The entrapment efficiency was 56%.

### EXAMPLE 6

(Entrapment of Thyrotropin Releasing Hormone)

Two ml of a 20% (w/v) aqueous emulsion of Span 20 (Sigma Chemical Co.) and 1 ml of an aqueous solution of thyrotropin releasing hormone (pGlu-His-Pro amide, TRH) were transferred to a glass vessel. To this vessel, 25 ml of 3% (w/v) poly(D,L-lactide-co-glycolide) (50/50 molar, i.v.−0.7) in dichloromethane were added. The mixture was homogenised for 1 minute using a Silverson homogeniser. Whilst continuing mixing, 20 ml of silicone oil (silicone oil DC200, −100 mPa.s) was added at a rate of 2 ml per minute using a syringe driver equipped with a 50-ml glass syringe. Hereafter, mixing was continued for 1 minute. The resulting mixture was then added to 200 ml stirred n-heptane. The stirring was continued for a minimum of 30 minutes, following which the microparticles were washed and collected as described in Example 4. Mean microparticle size was determined by laserlight diffraction. The entrapment of TRH in the microspheres was determined by dissolving the microspheres in dichloromethane, extracting TRH into an aqueous phase, and determining the extracted amount of TRH by high performance liquid chromatography. The results are shown in Table 5.

TABLE 5
(Entrapment of TRH in microspheres)

| Theoretical TRH | | Actual Loading | TRH Loading |
|---|---|---|---|
| loading (%) | Particle size (μm) | (% w/w) | efficiency (%) |
| 0.0 | 57 | 0 | — |
| 95 | 111 | 6.6 | 69 |
| 6.6 | 92 | 4.2 | 64 |
| 3.1 | 55 | 1.5 | 48 |
| 1.0 | 12 | 0.15 | 15 |

### EXAMPLE 7

(Entrapment of Insulin)

Two ml of a 20% (w/v) aqueous emulsion of span 20 (Sigma Chemical Co.) and 1 ml of an aqueous dispersion of insulin (Sigma) were transferred to a glass vessel. To this vessel, 25 ml of 3% (w/v) poly(D,L-lactide-co-glycolide) (50/50 molar, i.v.−0.7) in dichloromethane was added. Microparticles were prepared as described in Example 6. Microparticle size was determined by laserlight diffraction. The insulin entrapment was determined by dissolving the microparticles in DMSO and determining the amount of protein by the BCA method. The results are shown in Table 6.

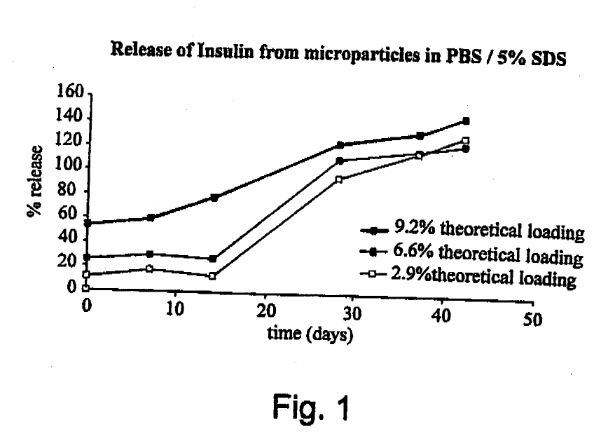

The release of insulin from these microparticles at 37° C. was determined in phosphate buffered saline containing 5% sodium dodecyl sulphate (SDS). The results are shown in FIG. 1. The active agent burst effect is apparent from the y axis increments.

TABLE 6

| | Theoretical in- | Particle size | Actual Load | Insulin loading |
|---|---|---|---|---|
| Polymer | sulin loading (%) | (μm) | (% w/w) | efficiency (%) |
| RG505 | 9.2 | 230 | 6.9 | 75 |
| RG505 | 6.6 | 291 | 5.4 | 82 |
| RG505 | 2.9 | 192 | 1.9 | 66 |

### EXAMPLE 8

(Comparison with/without Emulsifier)

1 ml of cytochrome C solution (25.32 mg) was mixed with approximately 1.5 g Span 80 (20% w/w) or 1.5 ml of water and the total aqueous phase adjusted to 3 g with water. 25 ml of 10% w/v RG503 (a 50:50 lactide/glycolide copolymer) solution in dichloromethane was then added, and the system mixed at 10000 rpm for 1 minute. 25 ml Silicone oil was added at 2 ml/minute whilst mixing following which mixing was continued for a further minute. The resulting system was poured into 200 ml of HPLC grade n-heptane and mixed for 1 hour. The supernatant was decanted off, 200 ml fresh n-heptane added and stirring continued overnight. Suspensions were filtered through a 500 μm mesh to remove gross precipitate, and microspheres were isolated by filtration onto a 1.2 μm cellulose ester membrane. A single batch of microspheres were produced on dry ice/methanol at −40° C.

Microsphere yield was determined gravimetrically, and the particle size distribution measured by laser light scattering (Malvern Mastersizer). Results are given in Table 7.

TABLE 7
Table: Summary Silicone Oil Viscosity Effect

| Silicone | | | | |
|---|---|---|---|---|
| Oil | | | Particle | Standard Division |
| Viscosity | Span 80 | Microsphere | Diameter | of Particle size |
| (mPa · s) | (g) | Yield (%) | (μm) | Distribution (μm) |
| 100 | 1.5029 | 92 | 128 | 86 |
| 378 | 1.5012 | 94 | 84 | 90 |
| 545 | 1.4994 | 89 | 177 | 81 |
| 1000 | 1.5004 | 87 | 66 | 45 |
| 100 | 0 | 0 | ND | ND |
| 378 | 0 | 0 | ND | ND |
| 545 | 0 | 0 | ND | ND |
| 1000 | 0 | 0 | ND | ND |
| 1000 cold | 0 | 0 | ND | ND |

ND: Not Determined

As can be seen, silicone oil viscosity has little influence on microsphere yield, although it does appear to influence the particle size distribution (albeit in an unpredictable manner). The most important effect observed was the inability to produce microspheres in the absence of emulsifier (a precipitate resulting), even with the most viscous oil at low temperatures. On first inspection this appears to conflict with the prior art which demonstrates particle production without surfactant. However, as previously discussed, the conventional surfactant-free process is only able to produce microspheres within a very narrow range of conditions, and it is likely that the systems manufactured above are outside this stability window. This further illustrates the wide stability window of the process of the invention.

### EXAMPLE 9

(Microsphere Production Stability Window)

A disadvantage of the conventional phase separation process is the relatively narrow range of conditions over which microparticles can be produced and the consequently restricted range of microparticles available. By introducing an emulsifier according to the present invention, microparticles can be produced over a wider range of conditions, with a consequently wider range of potential properties.

In order to illustrate these advantages, a series of microparticulate systems were manufactured with and without emulsifier and over a range of polymer concentrations, aqueous phase volumes and protein loading levels.

Span 80 was dispensed to a mixing vessel along with RG505 50/50 ratio lactide/glycolide copolymer (PLGA) dissolved in dichioromethane and cytochrome C solution. The composition of these systems is summarised in Table 8. Each system was homogenised whilst silicone oil (100 mPa.s) was added at 2 ml/min. samples were taken at designated intervals and dispersed into 100-200 ml n-heptane. The product that resulted was allowed to settle, rinsed with further heptane and recovered by filtration. Samples were dried and the nature of each product determined following the addition of water and brief sonication. The presence of a microparticulate product was indicated by formation a fine dispersion.

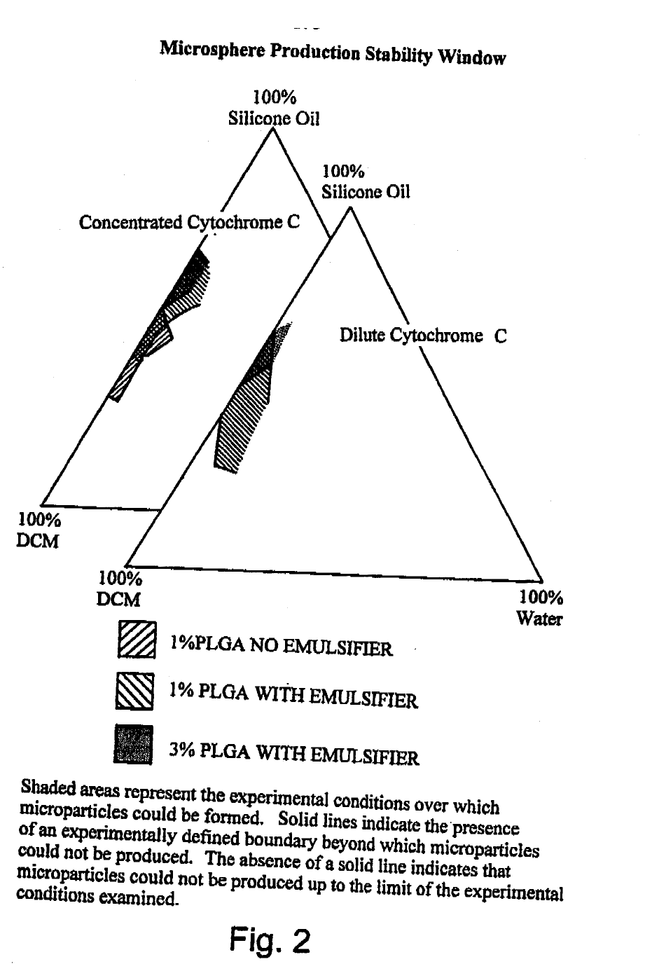

Conditions that resulted in the successful production of microparticles are illustrated in FIG. 2. It can be clearly seen that microparticles can be produced over a far wider range of conditions in the presence of an emulsifier. In particular, when high levels of cytochrome C were used it was impossible to produce particles at the higher level of polymer concentration in the absence of an emulsifier. Furthermore, the process was unable to form microparticles in the absence of an emulsifier when low levels of cytochrome C were used even at low polymer concentration.

TABLE 8

| | Polymer | Polymer | Aqueous | |
|---|---|---|---|---|
| Span 80 | Concentration | Solution | Phase Volume | Cytochrome |
| (g) | (%) | Volume (ml) | (ml) | C (mg) |
| 1.2050 | 1 | 100 | 1 | 2.60 |
| 1.2065 | 1 | 100 | 5 | 2.60 |
| 1.2157 | 1 | 100 | 10 | 2.60 |
| 1.2043 | 1 | 100 | 20 | 2.60 |
| 1.2022 | 3 | 100 | 1 | 2.60 |
| 1.2051 | 3 | 100 | 20 | 2.60 |
| 1.2042 | 3 | 100 | 10 | 2.60 |
| 0 | 1 | 100 | 1 | 2.60 |
| 0 | 1 | 100 | 5 | 2.60 |
| 0 | 1 | 100 | 1 | 25.03 |
| 0 | 1 | 100 | 10 | 25.03 |
| 1.2002 | 3 | 100 | 10 | 25.03 |
| 1.2039 | 3 | 100 | 20 | 25.03 |
| 1.2061 | 1 | 100 | 20 | 25.03 |
| 0 | 1 | 100 | 1 | 25.03 |
| 0 | 1 | 50 | 1 | 12.66 |
| 0.6025 | 1 | 50 | 1 | 12.66 |
| 0 | 1 | 50 | 6 | 12.66 |
| 0.6037 | 1 | 50 | 6 | 12.66 |
| 0 | 3 | 50 | 1 | 12.66 |
| 0.6060 | 3 | 50 | 1 | 12.66 |
| 0 | 3 | 50 | 6 | 12.66 |
| 0.6019 | 3 | 50 | 6 | 12.66 |

### EXAMPLE 10

(Efficiency of Active Entrapment)

This example relates to the entrapment of TRH as a function of polymer concentration. TRH is a very small molecule (3 amino acids) and is therefore more difficult to entrap than larger species. This experiment was performed using RG503 (a similar 50/50 co-polymer to RG505 but having a lower molecular weight) and a 75/25 lactide/glycolide co-polymer.

A failing of many microparticulate systems is rapid load release during the very early phase of the release profile. Known as burst release, this can be due to the presence in the formulation of free active compound, or the active's association with the particle surface rather than its entrapment. The importance of burst release arises due to the high potency of the active typically incorporated into microparticles and the risk of the development of toxic plasma levels.

It has been proposed that burst release can be attenuated by rinsing the product with an aqueous buffer prior to storage and administration. This, however, will result in the loss of a considerable quantity of often expensive active compound. It would be preferable for the formulation method to result in complete load entrapment.

TRH loaded microparticles were prepared. 3 g of an aqueous phase containing 400 mg Span 80 and 54 mg TRH was transferred into a mixing vessel along with 25 ml of lactide/glycolide copolymer in dichloromethane. This mixture was homogenised, and 20 ml silicone oil added under constant mixing. The resulting system was transferred into 200 ml n-heptane under stirring, the resulting product allowed to settle, supernatant decanted, further heptane added and stirring continued. Microparticles were recovered by filtration and dried.

Entrapped TRH was determined by HPLC following particle dissolution in dichloromethane and peptide extraction into an aqueous phase. Microparticles were then washed with demineralised water, in order to remove any non-entrapped or loosely associated active. Rinsed particles were re-dried and their TRH loading determined as above.

The results of this study are summarised in Table 9. These data demonstrate that 50:50 ratio polymer can result in the formation of microparticles that exhibit minimal burst release, particularly if higher concentrations (e.g. 10% w/w) of polymer solution are utilised in manufacture.

Furthermore, this example demonstrates the ability of the phase separation process of the invention to promote high entrapment efficiency.

TABLE 9

| | Polymer | | | | |
|---|---|---|---|---|---|
| Polymer | Concentration | Loading (% w/w) | | Loading Efficiency | |
| Ratio (L:G) | (% w/w) | Unrinsed | Rinsed | Unrinsed | Rinsed |
| 75:25 | 1 | 6.61 | 0.03 | 30.6 | 0.2 |
| 75:25 | 5 | 0.51 | 0.03 | 11.8 | 0.7 |
| 75:25 | 10 | 0.99 | 0.01 | 45.9 | 0.5 |
| 50:50 | 1 | 4.17 | 0.17 | 19.3 | 0.8 |
| 50:50 | 5 | 1.78 | 0.61 | 41.2 | 14.1 |
| 50:50 | 10 | 1.31 | 1.34 | 60.7 | 62.1 |

### EXAMPLE 11

(Alternative Polymers)

800 mg Span 80 was dispensed to a mixing vessel along with 25 ml of polymer solution. Demineralised water was added and the system homoqenised. Silicone oil was added under constant mixing and the resulting system decanted into 200 ml n-heptane and stirred. Stirring was halted, allowing the product to settle. The supernatant was decanted, fresh n-heptane added, and stirring continued. Microparticles were then collected on a membrane filter and allowed to dry overnight. Microparticle diameters were determined by laser light diffraction. The results of this study are reported in Table 10 and indicate that the process described is flexible with respect to the polymers that can be utilised.

TABLE 10

| | Concentration | Silicone oil | Water | Particle |
|---|---|---|---|---|
| Polymer | (% w/v) | Volume (ml) | Volume (ml) | Diameter (μm) |
| PCL | 6 | 10 | 2 | 69 |
| PCL | 6 | 20 | 2 | 271 |
| PCL | 3 | 10 | 2 | 168 |
| PCL | 3 | 20 | 2 | 97 |
| PVC | 3 | 10 | 3 | 103 |
| PVC | 3 | 20 | 3 | 70 |

PCL: Poly(caprolactone)
PVC: Poly(vinylchloride)

### EXAMPLE 12

(Alternative Phase Separating Agent)

800 mg Span 80 was dispensed into a mixing vessel along with 3 ml demineralised water and 25 ml of 3% (w/v) poly(D,L-lactide-co-glycolide) (50:50 molar ratio, i.v. −0.5) in dichloromethane. The system was then homogenised and 20 ml of coacervation agent added at a rate of 2 ml per minute under constant mixing. The resulting mixture was transferred into 200 ml n-heptane, and stirred. Stirring was halted, allowing microparticles to settle. The supernatant was decanted, 200 ml fresh n-heptane added, and stirring continued. The microparticulate product was then collected on a membrane filter and allowed to dry overnight.

Microparticle size distributions were determined by laser light scattering, and the results summarised in Table 11. These data indicate that the phase separation process here described is not limited by the use of silicone oil.

TABLE 11

| Coacervation | Particle |
|---|---|
| Agent | Diameter (μm) |
| Silicone oil | 27 |
| Mineral oil | 131 |

### EXAMPLE 13

(Leuprolide Entrapment)

800 mg Span 80 was transferred into a mixing vessel along with 2 ml of an aqueous phase containing 20 mg leuprolide and 40 ml 3% (w/v) poly(D,L-lactide-co-glycolide) (50:50 molar ratio, i.v. −0.7) in dichloromethane. This mixture was homogenised and 30 ml silicone oil (100 mPas) added under constant mixing. The resulting system was transferred into n-heptane and stirred. The resulting product was allowed to settle, the supernatant decanted, fresh heptane added and mixing continued. Microparticles were recovered by filtration. Manufacture was carried out in triplicate and the batches combined.

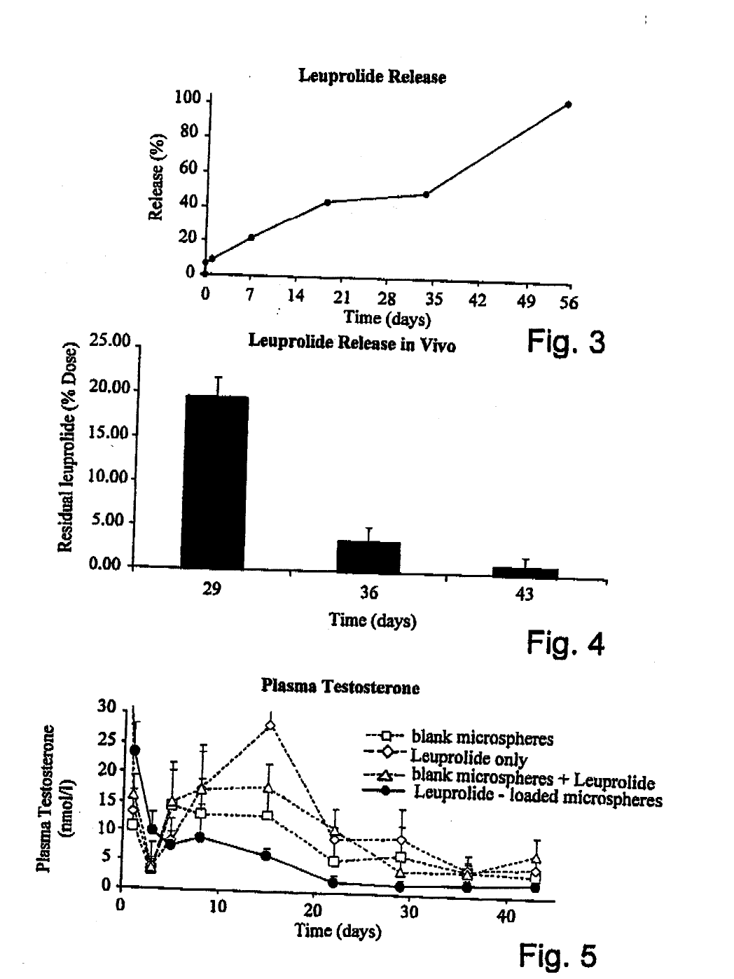

The microparticle size distribution was determined by laser light diffraction as 40 μm. The leuprolide content of the microparticles was determined as 0.71% w/w by HPLC following particle dissolution and extraction of the peptide into an aqueous phase. Leuprolide release was determined by re-suspending a quantity of microparticulate material in 10 mm MES buffer (pH 7.2) and incubating the system at 37° C. At the required time, samples were drawn, centrifuged and the leuprolide present in the aqueous supernatant determined by UV absorbance. This data is summarised in FIG. 3, which demonstrates the controlled release or leuprolide from microparticles over a period of 55 days.

### EXAMPLE 14

(In Vivo Release)

Four groups of 15 male Sprague-Dawley rats received, leuprolide loaded microparticles (manufactured as detailed in Example 13), blank microparticles, blank microparticles with free leuprolide or free leuprolide according to the dose schedule given in Table 12. Administration was by subcutaneous injection into the right flank, and injection sites were marked with a marker pen in order to facilitate their excision at necropsy.

At defined times, 5 members from each group were sacrificed, and the injection sites removed and stored at −40° C. until analysed.

Injection site tissue was finely shredded and homogenised in a mixture consisting of 10 ml hexane and 5 ml phosphate buffer. Following homogenisation, the homogeniser head was rinsed with phosphate buffer, rinsing's being added to the sample. Samples were centrifuged, the hexane removed, 5 ml dichloromethane added and then shaken overnight. Samples were then extracted with phosphate buffer several times and the leuprolide content of the combined extracts determined by HPLC.

The results of this study are presented in FIG. 4 and demonstrate controlled release of leuprolide from the injection site of rats treated with leuprolide loaded microparticles over 43 days. No leuprolide was recovered at any time from injection sites of rats treated with blank microparticles, free leuprolide or free leuprolide co-administered with blank microparticles.

TABLE 12

| | Blank | Leuprolide | |
|---|---|---|---|
| Group | Microparticles | Microparticles | Leuprolide |
| 1 | 610 mg/kg | — | — |
| 2 | — | — | 3 mg/kg |
| 3 | 610 mg/kg | — | 3 mg/kg |
| 4 | — | 610 mg/kg | — |

### EXAMPLE 15

(Leuprolide Bioactivity)

An important aspect of any formulation intended for the delivery of peptide based pharmaceuticals is the retention of the active's activity following formulation and administration. This is especially true for a controlled release system that is intended to release active over a prolonged period, typically months.

It is well known that the continued presence of a LHRH analog, for example leuprolide, results in the suppression of plasma testosterone levels following an initial surge. This activity affords a convenient method for the assessment of LHRM bio-activity, and was used to assess the activity of leuprolide formulated into microspheres as detailed in Example 13.

Rats were treated with leuprolide loaded microparticles, blank microparticles, free leuprolide and free leuprolide co-administered with blank microparticles as outlined in Example 14. Blood samples were drawn periodically, and plasma testosterone levels determined by radioimmunoassay.

The results of this study are summarised in FIG. 5. After day three, rats which received leuprolide loaded particles exhibited testosterone levels that were consistently lower than all other groups. Furthermore, these levels were found to be significantly lower on days 5, 15, 22 and 33 than for rats treated with a mixture of blank microparticles and free leuprolide.

These data clearly illustrate the ability of leuprolide loaded microparticles to suppress plasma testosterone, thus demonstrating that leuprolide remains bioactive following formulation into microspheres. Furthermore, the presence of active leuprolide is suggested up to day 43 therefore confirming the products sustained,release character.

### EXAMPLE 16

(Local Irritation

Leuprolide loaded microparticles were prepared by the phase separation method. 3 ml of an aqueous phase containing 400 mg span 80 and 20 mg leuprolide was transferred to a mixing vessel to which was added 25 ml of 1.5% (w/v) poly(D,L-lactide-co-glycolide) (50:50 molar ratio, i.v. −0.7) in dichloromethane. This mixture was homogenised and 25 ml silicone oil added under constant mixing. The resulting system was transferred into 200 ml n-heptane under stirring and the resulting product allowed to settle, supernatant decanted, further n-heptane added and stirring continued. Microparticles were dried and their particle size determined by laserlight diffraction. Entrapped leuprolide was determined by HPLC following particle dissolution in dichloromethane and the extraction of peptide into an aqueous phase. Microparticles were found to have a diameter of 31 μm and a leuprolide content of 0.75% w/w.

Blank microparticles were prepared by the phase separation method. 3 ml of an aqueous phase containing 400 mg Span 80 was transferred to a mixing vessel to which was added 25 ml of 3% (w/v) poly(D,L-lactide-co-glycolide) (50:50 molar ratio, i.v. −0.7) in dichloromethane. This mixture was homogenised and 20 ml silicone oil added under constant mixing. The resulting system was transferred into 200 ml n-heptane under stirring and the resulting product allowed to settle, supernatant decanted, further n-heptane added and stirring continued. Microparticles were dried and were found to have a diameter of 45 μm and to contain no entrapped leuprolide.

Both blank and leuprolide loaded microparticles were dispersed in a buffer consisting of 1 sodium carboxymethyl cellulose, 0.2% Tween 80, 0.14% methyl p-hydroxybenzoate, 0.014% propyl p-hydroxybenzoate and 5% sorbitol. Groups of 5 male Sprague-Dawley rats were dosed with 125 mg/kg of microparticles, either blank or leuprolide-loaded, 7.5 mg/kg leuprolide or an appropriate volume of vehicle. Rats were sacrificed after 28 days, the injection sites excised, and subjected to histopathological investigation.

Some animals injected with microspheres, whether blank or loaded with Leuprolide, developed a localised, well defined, chronic, granulomatous inflammatory reaction at the site of deposition, graded mild; the reaction being typical of a response to a foreign body. There was no evidence of a more diffuse inflammatory reaction in the peripheral tissues.

These results suggest that microparticles manufactured by the method here described are bio-compatible and do not result in any irritation other than that expected from the injection of any foreign body.

### EXAMPLE 17

(Residual Substances)

The quantity of residual manufacturing components remaining in batches of microparticles manufactured as described above were determined.

Silicone oil was determined by NMR spectroscopy. Standards were prepared by dispensing 50 mg of PLGA into glass vials and dissolving this material in dichloromethane laced with known quantities of silicone oil (100 mPas). Following polymer dissolution, solvent was removed by evaporation, and the standards vacuum desiccated. 1 ml deuterated chloroform was then added, the material shaken overnight, and the ¹H NMR spectrum of each standard determined. Samples were handled in a similar way except that the chloroform used to dissolve microparticles was silicone oil free. The silicone oil/solvent proton was calculated from the ¹H spectrum. This ratio was used to construct a liner calibration series for standards, from which unknown's could be determined. The results of this study are summarised on Table 13 and indicate that the process described above has the potential to produce microparticles with very low levels of residual silicone oil.

Residual dichloromethane and n-heptane were determined by GC. Samples were weighed into glass vials and dissolved in 1,4-dioxane. Iso-octane, containing 2-butoxyethanol as an internal standard, was then added to each vial in order to precipitate the polymer. Precipitate was removed by centrifugation and the samples analysed by GC(SGE BPX5 25 m×0.32 mm,1 μl split injection (20:1 split) at 280° C., Helium, 2 ml/min). Calibration was carried out using heptane and DCM dissolved in 1,4-dioxane and isooctane (1:2) with 2-butoxyethanol as an internal standard. The results of this study are summarised in Table 13 and indicate that the microsphere production method here described has the potential to produce product with very low levels of residual solvent.

Residual Span 80 was determined by UV absorbance (A₂₃₂) following dissolution or samples in acetonitrile, and solvent extraction of the emulsifier. Quantification was carried out by reference to the absorbencies obtained for known standards. The results of this study are summarised in Table 13 and indicate the microparticles produced by this method are associated with quantity of residual emulsifier. This may act to the benefit of the product since it may increase surface hydrophilicity and therefore aid particle wetting and redispersion.

TABLE 13

| Contaminant | Range (% w/w) |
|---|---|
| Silicone Oil | 0.025-0.316 |
| Span 80 | 1.9-2.0 |
| Heptane | 0.32-4.10 |
| Dichloromethane | <0.03-2.98 |

1. A method of producing microparticles, which comprises
incorporating an emulsifier in an aqueous liquid and/or a non-aqueous solution containing a polymer;
forming a dispersion of the aqueous liquid in the non-aqueous solution; and
agitating the dispersion and adding thereto a non-solvent for the polymer so as to form polymer microparticles.

2. A method according to claim 1 wherein the emulsifier is included in the aqueous liquid.

3. A method according to any preceding claim wherein the emulsifier is a non-ionic surfactant.

4. A method according to any preceding claim wherein the emulsifier is present in an amount of up to 60% by weight of the aqueous liquid or non-aqueous solution.

5. A method according to claim 4 wherein the emulsifier is present in an amount of up to 30% by weight.

6. A method according to any preceding claim wherein the emulsifier is present in the aqueous liquid or non-aqueous solution in an amount of at least 1% by weight.

7. A method according to any preceding claim wherein the emulsifier has an HLB value of 2 to 9.

8. A method according to any preceding claim wherein the aqueous liquid is from 5 to 50% by weight of the dispersion.

9. A method according to any preceding claim wherein the polymer is a polylactide-co-glycolide copolymer.

10. A method according to claim 9 wherein the ratio of lactide to glycolide is from 75:25 to 50:50 respectively.

11. A method according to any preceding claim wherein the polymer is present in the non-aqueous solution in an amount of 0.5 to 10% by weight.

12. A method according to any preceding claim wherein the non-aqueous solvent is dichloromethane.

13. A method according to any preceding claim wherein the non-solvent has a viscosity of less than 500 mPa.

14. A method according to claim 13 wherein the viscosity is 50-150 mPa.

15. A method according to any preceding claim wherein a pharmaceutically active agent is present in the aqueous liquid.

16. A method according to claim 15 wherein the active agent is a protein or peptide.

17. A method according to any preceding claim wherein the loading efficiency (as defined herein) is at least 60%.

18. A microparticle which comprises a pharmaceutically active agent, residual emulsifier and a pharmaceutically acceptable polymer.

19. A microparticle according to claim 18 wherein the residual emulsifier is present in an amount of up to 5% by weight.

20. A microparticle according to claim 18 or 19 which is substantially free of hydrophobic non-solvent.

21. A microparticle according to claim 18, 19 or 20 having a particle size of 10-300 microns.
